(19) United States
(12) Patent Application Publication
(10) Pub. No.: US 2025/0065044 A1
(43) Pub. Date: Feb. 27, 2025

(54) DEVICE FOR AUTOMATIC DETECTION OF IV DROP FACTOR, SYSTEMS INCLUDING THE SAME, AND METHODS OF USE THEREOF

(71) Applicant: CareFusion 303, Inc., San Diego, CA (US)

(72) Inventor: Jithendra Kumar Sathyanarayana Naidu, Singapore (SG)

(21) Appl. No.: 18/695,642

(22) PCT Filed: Feb. 24, 2023
(86) PCT No.: PCT/CN2023/013872

§ 371 Date: Mar. 26, 2024

(51) Int. Cl. A61M 5/168; A61M 5/162

(52) U.S. Cl. CPC A61M 5/1689; A61M 5/162; A61M 2205/18; A61M 2205/3306; A61M 2205/502; A61M 2205/60

(57) ABSTRACT

A device for detecting a drop-factor identifier of an intravenous (IV) tubing set includes a housing having a recess, a drop-factor sensor, and a drip-rate sensor. The recess in the housing is configured to couple the device to a spike of an IV tubing set. The recess is also configured to receive a portion of a drip chamber connected to the spike while the device is coupled to the spike. The drop-factor sensor is configured to detect a drop-factor identifier of the IV tubing set while the device is coupled to the spike via the recess. The drop-factor identifier indicates a drop factor of the IV tubing set. The drip-rate sensor is configured to detect a drip rate of the IV tubing set while the device is coupled to the spike via the recess.

### BACKGROUND

For generations, health care facilities have relied on intravenous (IV) therapy for delivering hydration, nutrition, and medication to patients throughout the world. An IV therapy requires little more than a fluid bag, an IV tubing set, and a catheter. And yet, despite its simplicity, IV therapy is not without issues.

Oftentimes, IV therapy requires that an IV fluid be administered at a particular flow rate. Without an electronic infusion pump to administer the IV fluid according to a programmed therapy, achieving a particular flow rate may require determining the drop factor of the IV tubing set, determining the drip rate of the IV tubing set, and performing on-the-fly mathematics. Moreover, ensuring that the IV therapy continues at the necessary flow rate may require repeatedly checking on the IV tubing set.

These requirements introduce the possibility for human error into IV therapy, especially considering it is performed in high-pressure medical environments. Accordingly, there is a need for a device capable of determining the drop factor, the drip rate, and the flow rate of an IV tubing set.

### SUMMARY

According to various aspects of the subject technology, a device for detecting a drop-factor identifier of an intravenous (IV) tubing set includes a housing having a recess, a drop-factor sensor, and a drip-rate sensor. The recess is configured to couple the device to a spike of the IV tubing set. The recess is also configured to receive a portion of a drip chamber connected to the spike while the device is coupled to the spike. The drop-factor sensor is configured to detect the drop-factor identifier of the IV tubing set while the device is coupled to the spike via the recess, where the drop-factor identifier indicates a drop factor of the IV tubing set. And the drip-rate sensor is configured to detect a drip rate of the IV tubing set while the device is coupled to the spike via the recess.

According to various aspects of the subject technology, a system for monitoring a flow rate of an IV tubing set includes a device and a display. The device includes a housing having a recess, a drop-factor sensor, and a drip-rate sensor. The recess is configured to couple the device to a spike of the IV tubing set and receive a portion of a drip chamber connected to the spike while the device is coupled to the spike. The drop-factor sensor is configured to detect a drop-factor identifier of the IV tubing set, where the drop-factor identifier indicates a drop factor of the IV tubing set. And the drip-rate sensor is configured to detect a drip rate of the IV tubing set. The display is configured to display the flow rate of the IV tubing set, wherein the flow rate is based on the drop factor and the drip rate.

According to various aspects of the subject technology, a method of producing a device for detecting a drop-factor identifier of an IV tubing set includes providing a housing having a recess configured to couple the device to a spike of the IV tubing set and receive a portion of a drip chamber connected to the spike while the device is coupled to the spike. The method also includes providing a drop-factor sensor configured to detect a drop-factor identifier of the IV tubing set while the device is coupled to the spike via the recess, where the drop-factor identifier indicates the drop factor of the IV tubing set. Additionally, the method includes providing a drip-rate sensor configured to detect a drip rate of the IV tubing set while the device is coupled to the spike via the recess.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Detailed Description below, in conjunction with the Figures. Like reference numerals refer to corresponding parts throughout the Figures and Description.

FIG. 1 illustrates an example device for detecting a drop-factor identifier and a drip rate of an IV tubing set, according to aspects of the subject technology.

FIGS. 2A-2C illustrate the example device being coupled to an example IV tubing set that includes a spike and a drip chamber, according to aspects of the subject technology

FIG. 3 illustrates an example system for monitoring a flow rate of a first IV tubing set and a second IV tubing set, according to aspects of the subject technology.

FIG. 4 is a conceptual diagram illustrating an example electronic system for detecting a drop-factor identifier of an IV tubing set or monitoring a flow rate of an IV tubing set, according to aspects of the subject technology.

### DETAILED DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In some instances, well-known methods, procedures, components, circuits, and networks have not been described in detail so as not to unnecessarily obscure aspects of the implementations.

As noted above, there is a need for a device capable of determining the drop factor, the drip rate, and the flow rate of an IV tubing set. The present disclosure describes devices and systems capable of determining these metrics at a significantly lower price point than that of an infusion pump. The present disclosure also describes methods of using and manufacturing the aforesaid devices and systems.

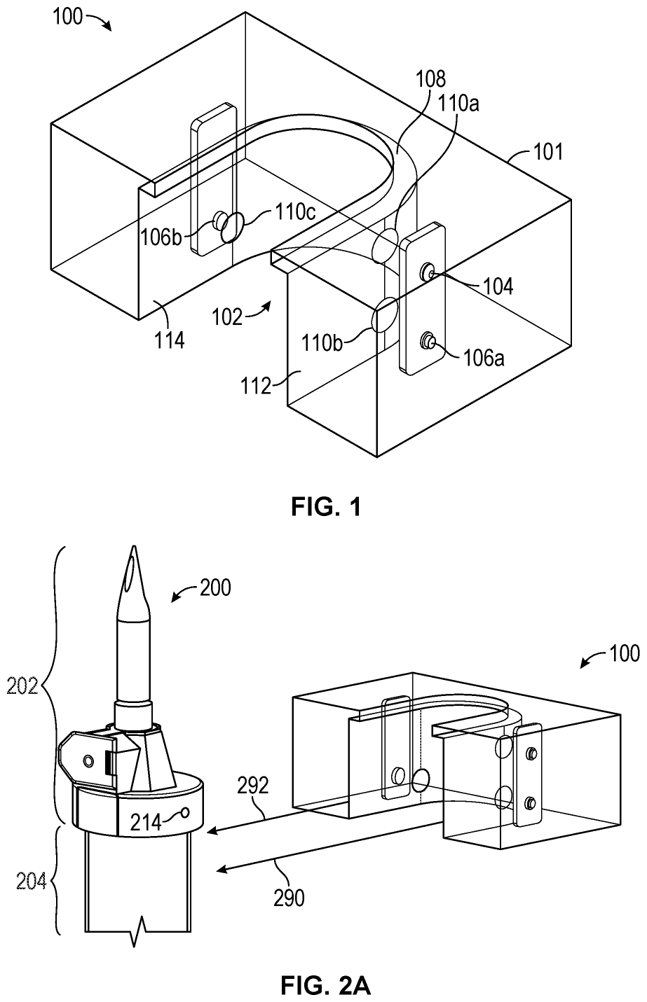

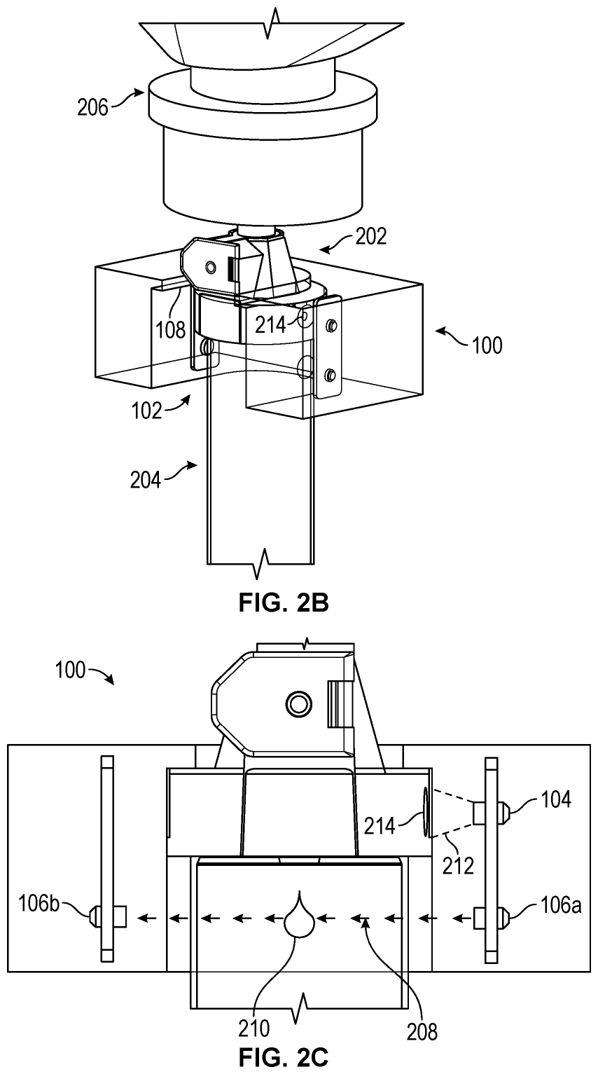

FIG. 1 illustrates an example device 100 for detecting a drop-factor identifier (e.g., drop-factor identifier 214 of FIGS. 2A-2C) and a drip rate of an IV tubing set (e.g., IV tubing set 200 of FIGS. 2A-2C), according to aspects of the subject technology. In some implementations, the device 100 can also determine the drop factor of the IV tubing set based on the drop-factor identifier. For example, the device 100 may include a processor configured to determine the drop factor of the IV tubing set. Additionally, in some implementations, the device 100 can determine the flow rate of the IV tubing set based on the drop factor and the drip rate.

For a given IV tubing set, “drop factor” refers to the number of drops through the IV drip chamber that are needed to produce one milliliter of IV fluid. As an example, if the drop factor of an IV tubing set is 20 gtts/mL, then each drop that drips through the drip chamber will be 1/20^{th} of a milliliter (i.e., 0.05 mL). Common drop factors include 10, 15, 20, and 60 gtts/mL. Further, the drop factor of an IV tubing set can be used with the drip rate of the IV tubing set to determine the flow rate thereof. For example, the following formula may be used to determine the flow rate of an IV tubing set based on dividing the drop factor by the drip rate:

$$F = ( 60 ) \frac{R}{F}$$

where F is the flow rate in milliliters per hour (mL/hr), R is the drip rate in drops per minute (gtts/min), and F is the drop factor in drops per milliliter (gtts/mL).

As illustrated, the device 100 includes a housing 101 with a recess 102 formed within the housing 101. In some implementations, the recess 102 is configured to couple the device 100 to an IV tubing set. For example, as depicted in FIGS. 2A-2C, the device 100 can be coupled to a spike 202 of an IV tubing set 200 via the recess 102 of the device 100. To enhance the coupling between the device 100 and the IV tubing set, the recess 102 may include a shelf 108. In some implementations, the shelf 108 is configured to sit on top of a relatively-horizontal portion of the IV tubing set while the device 100 is coupled to the IV tubing set. In some implementations, the shelf is formed from (e.g., part of) the housing 101 together with the recess 102. An example of this is depicted in FIGS. 2B and 2C, where the shelf 108 sits on top of a base of a spike 202 of the IV tubing set 200.

In addition to the recess 102 and the shelf 108 thereof, the present disclosure also contemplates other components for coupling the device 100 to the IV tubing set. For example, the device 100 may include an adhesive or a magnet (e.g., within the housing 101 and/or the recess 102) for coupling the device 100 to the IV tubing set. As another example, the device 100 may include high-friction components (e.g., rubber pads positioned in the recess 102) for coupling the device 100 to the IV tubing set.

The recess 102 is semicylindrical and includes a first side 112 and a second side 114 opposite the first side. The first side 112 includes a drop-factor sensor 104 positioned therein. In some implementations, the drop-factor sensor 104 is configured to detect a drop-factor identifier (e.g., drop-factor identifier 214 of FIGS. 2A-2C) of an IV tubing set (e.g., IV tubing set 200 of FIG. 2A) while the device 100 is coupled to the IV tubing set. For example, the drop-factor identifier can be affixed to the portion of the IV tubing set received by the recess 102 of the device 100. In some implementations, the device 100 is configured to determine the drop factor of the IV tubing set based on the drop-factor identifier (e.g., via a lookup table).

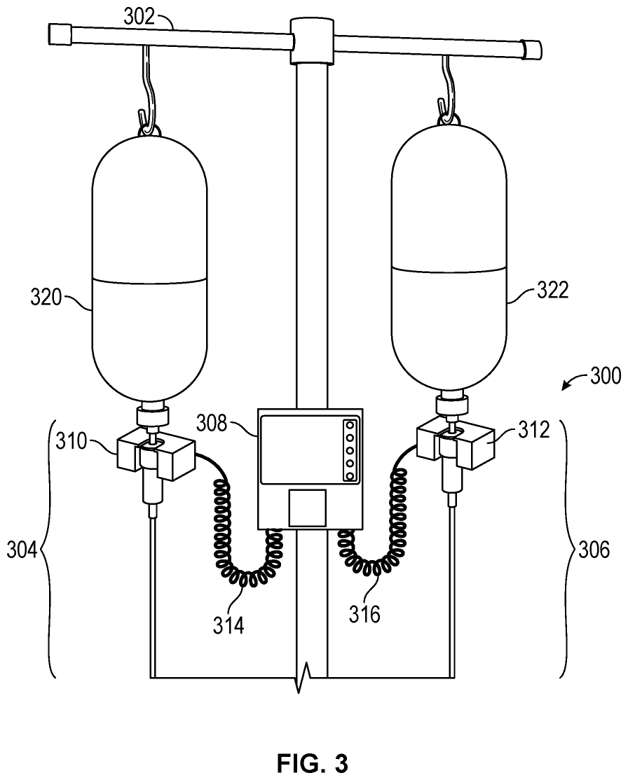

In some implementations, the device 100 includes an onboard display. For example, the onboard display can be configured to display the drop factor of the IV tubing set (e.g., determined based on the drop-factor identifier) and prompt a clinician to indicate whether the drop factor displayed is correct (e.g., whether it matches the actual drop factor of the IV tubing set). As another example, the onboard display can be configured to display the flow rate of the IV tubing set (e.g., after receiving a confirmation that the drop factor displayed is correct). In some implementations, the device 100 is connected to an external display, such as the display device 308 discussed below with respect to FIG. 3.

The first side 112 of the recess 102 also includes a transmitter 106a positioned therein. The transmitter 106a sits opposite a receiver 106b that is positioned in the second side 114 of the recess 102. Together, the transmitter 106a and the receiver 106b comprise a drip-rate sensor 106a-b. In some implementations, the drip-rate sensor 106a-b is configured to detect a drip rate of an IV tubing set (e.g., IV tubing set 200) while the device 100 is coupled to the IV tubing set. The functionality of the drip-rate sensor, as well as that of the aforementioned drop-factor sensor, is discussed in more detail below with respect to drip-rate sensor 106a-b (see FIG. 2C).

The example device 100 also includes holes 110a-c. Each of the holes 110a-c is associated with one of the drop-factor sensor 104, the transmitter 106a, and the receiver 106b. In some implementations, hole 110a allows the drop-factor sensor 104 to view or otherwise access a portion of the IV tubing set through the hole 110a. In some implementations, hole 110a allows the drop-factor sensor 104 to send or receive a signal (e.g., a near-field-communication (NFC) signal) to or from the IV tubing set. In some implementations, holes 110b-c allow a signal from the transmitter 106a to pass to the receiver 106b without obstruction.

The present disclosure recognizes that the holes 110a-c may be unnecessary. For example, the holes 110a-c may be unnecessary if the drop-factor sensor 104 or the drip-rate sensor 106a-b are located on an outward-facing portion of the recess 102. As another example, the holes 110a-c may be unnecessary if the drop-factor sensor 104 or the drip-rate sensor 106a-b can transmit or receive respective signals through the housing of the device 100.

FIGS. 2A-2C illustrate the example device 100 coupled to an example IV tubing set 200 that includes a spike 202 and a drip chamber 204, according to aspects of the subject technology. In the illustrated implementation, the recess 102 of the device 100 is configured to couple the device 100 to the spike 202 of the IV tubing set 200. Coupling the device 100 to the IV tubing set 200 (e.g., the spike 202 of the IV tubing set 200) may include, for example, bringing the device 100 into contact with the IV tubing set 200 such that the IV tubing set 200 is received into the recess 102 of the device 100, as indicated by arrows 290 and 292.

In FIG. 2B, the device 100 is coupled to the IV tubing set 200 via the recess 102. While the device 100 is coupled to the IV tubing set 200, the recess 102 circumscribes a portion of the drip chamber 204 and the shelf 108 rests atop a base portion of the spike 202. The shelf may serve, for example, to strengthen the coupling between the device 100 and the IV tubing set 200. Additionally, or in the alternative, the coupling may be strengthened by decreasing a width of the recess 102 such that the width of the recess 102 is less than a diameter of the spike 202 or a diameter of the drip chamber 204. In this manner, the recess 102 can be configured to grip the spike 202 or the drip chamber 204 while the device 100 is coupled to the IV tubing set 200. Further, as mentioned above, high-friction pads (e.g., rubber pads) can be added to the recess 102 to improve the coupling between the device 100 and the IV tubing set 200.

Also illustrated in FIG. 2B is an IV bag 206 containing an IV fluid (e.g., water, medication). The spike 202 of the IV tubing set 200 is connected to the IV bag 206, for example by inserting the spike 202 into the IV bag 206. While the spike 202 is connected to the IV bag 206, the IV fluid can flow down through the spike 202 and drip into the drip chamber 204.

Turning now to FIG. 2C, the functionality of the drop-factor sensor 104 and the drip-rate sensor 106a-b is highlighted by the cross-sectional view of the device 100 and the IV tubing set 200. As noted above, in some implementations, the drop-factor sensor 104 is configured to detect a drop factor of the IV tubing set 200 while the device 100 is coupled to the IV tubing set 200. This is illustrated by the beam 212 emitting from the drop-factor sensor 104 towards the IV tubing set 200. Though, it should be noted that the drop-factor sensor 104 need not emit a beam in order to detect the drop-factor of the IV tubing set 200.

For example, the drop-factor identifier 214 may be a visual identifier (e.g., printed on the IV tubing set 200), a magnetic identifier (e.g., embedded into the IV tubing set 200), or a wireless identifier. Accordingly, in implementations where the drop-factor identifier 214 is a visual identifier (e.g., a color, a shape, a symbol, or an encoded glyph, such as a QR code), the drop-factor sensor may include an optical sensor configured to detect a property of the visual identifier. In implementations where the drop-factor identifier 214 is a magnetic identifier (e.g., shaped metal affixed to or embedded within the portion of the drip chamber), the drop-factor sensor may include a magnetic sensor configured to detect a property of a magnetic field proximate to the magnetic identifier. And in implementations where the drop-factor identifier 214 is a wireless identifier (e.g., a near-field-communication tag), the drop-factor sensor may include a wireless sensor configured to detect a property of the wireless identifier (e.g., a signal transmitted by the wireless identifier).

As noted above with respect to FIG. 1, the drop-factor identifier 214 can be used to determine the drop factor of the IV tubing set 200. For example, a lookup table (e.g., stored in a memory) can be used to determine the drop factor based on using the drop-factor identifier 214 as an index into the lookup table. As another example, the drop-factor identifier 214 can be provided to a server or a database that, in response, returns the corresponding drop factor.

Similar to the drop-factor sensor 104, in some implementations, the drip-rate sensor 106a-b can be configured to detect a drip rate of the IV tubing set 200 while the device 100 is coupled to the IV tubing set 200. This is illustrated by the drop 210 of IV fluid and the signal path 208 between the transmitter 106a and the receiver 106b of the drip-rate sensor 106a-b. As illustrated, as the drop 210 of IV fluid falls through the drip chamber 204, it passes through the signal path 208, interrupting it temporarily. The drip-rate sensor 106a-b can then detect the interruption caused by the drop 210 of IV fluid.

In this manner, the drip-rate sensor 106a-b is configured to detect drops of IV fluid falling through the drip chamber 204. As discussed above, drop data captured by the drip-rate sensor 106a-b can be used to determine a drip rate of the IV tubing set by dividing the number of drops detected in an amount of time by the amount of time over which the drops were detected (e.g., 60 gtt/min).

FIG. 3 illustrates an example system 300 for monitoring a flow rate of a first IV tubing set 304 and a second IV tubing set 306, according to aspects of the subject technology. The system 300 includes a first device 310 (e.g., the device 100 of FIGS. 1-2C) coupled to the first IV tubing set 304 (e.g., the IV tubing set 200 of FIGS. 2A-2C) and a second device 312 coupled to the second IV tubing set 304. As illustrated, both of the first and second IV tubing sets 304 and 306 are connected to respective IV bags 320 and 322, which are suspended from an IV pole 302.

In some implementations, the first and second devices 310 and 312 are coupled, respectively, to the first and second IV tubing sets 304 and 306, as described above with respect to the device 100 and the IV tubing set 200 (see FIGS. 2A-2C). Further, in some implementations, each of the first and second devices 310 and 312 include drop-factor and drip-rate sensors, such as the aforementioned drop-factor sensor 104 and the drip-rate sensor 106a-b (see FIGS. 1 and 2C). Accordingly, the first and second devices 310 and 312 can each be configured to detect a drop-factor identifier (e.g., the drop-factor identifier 214 of FIGS. 2A-2C) and a drip rate of the first and second IV tubing sets 304 and 306, respectively.

In addition to the first and second devices 310 and 312, the system 300 also includes a display device 308. The display device 308 of the example system 300 is coupled to the first and second devices 310 and 312 via respective first and second wired connections 314 and 316. Additionally, the display device 308 is coupled to the IV pole 302. In this manner, the display device 308 can send data to, and receive data from, the first and second devices 310 and 312 via the first and second wired connections 314 and 316. The display device 308 can also display data regarding therapies administered via the first and second IV tubing sets 304 and 306, such as the flow rates thereof.

For example, the display device 308 can receive a drop-factor identifier and a drip rate of the first IV tubing set 304 from the first device 310. In this example, the display device 308 can determine the drop factor of the first IV tubing set 304 based on the drop-factor identifier (e.g., via a lookup table) after receiving it from the first device 310. Then, the display device 308 can determine a flow rate of the first IV tubing set 304 based on the drop factor and the drip rate.

As another example, the display device 308 can receive a drop factor and a drip rate of the second IV tubing set 306 from the second device 312. In this second example, the display device 308 can determine a flow rate of the second IV tubing set 306 based on the drop factor and the drip rate.

In some implementations, the display device 308 comprises an electronic device that is not coupled to the IV pole 302. For example, the display device 308 can be a mobile device, such as a smartphone, a tablet computer, or a laptop computer. As another example, the display device 308 can be a desktop computer. Accordingly, in some implementations, the display device 308 is configured to communicate wirelessly (e.g., via Bluetooth or Wi-Fi) with the first and second devices 310 and 312. In this manner, the display device 308 can receive information regarding the first and second IV tubing sets 304 and 306 without being physically tethered to the first and second devices 310 and 312 (e.g., via the first and second wired connections 314 and 316).

Additionally, in some implementations, the display device 308 includes an input interface (e.g., a touchscreen, physical buttons) that allow a clinician to input information into the display device 308. For example, the display device 308 may determine that the drop factor of the first IV tubing set 304 is 60 gtts/mL. The display device 308 may then prompt the clinician to confirm that the determined drop factor is correct by inputting a confirmation via the input interface.

As another example, the input interface of the display device 308 may allow the clinician to input a desired flow rate (e.g., 100 mL/hr), as well as a tolerance for deviation from the flow rate (e.g., ±5%). In some implementations, the display device 308 or the first or second devices 310 and 312 are configured to alert the clinician if the determined flow rate of the respective IV tubing set (e.g., the first or second IV tubing set 304 or 306) deviates from the desired flow rate (e.g., deviates to a degree that is greater than the input tolerance for deviation).

Similarly, in some implementations, the first device 310, the second device 312, or the display device 308 is configured to determine a starting flow rate (e.g., after receiving a confirmation of infusion, after the first device 310 or the second device 312 is coupled, respectively, to the first or second IV tubing sets 304 or 306). Additionally, in some implementations, the first device 310, the second device 312, or the display device 308 may be configured to determine a current flow rate and trigger an alarm in accordance with a determination that a difference between the current flow rate and the starting flow rate satisfies a deviation threshold.

In some implementations, the first device 310, the second device 312, or the display device 308 is configured to determine (e.g., redetermine) the current flow rate periodically. The frequency at which the current flow rate is determined may be based on the current flow rate of the IV tubing set. For example, if the flow rate is high, the frequency at which the current flow rate is determined may also be high. As another example, if the flow rate is low, the frequency at which the current flow rate is determined may be low, as well. Moreover, in some implementations, the first device 310, the second device 312, or the display device 308 is configured to periodically determine (e.g., redetermine) whether a difference between the current flow rate and a target flow rate (e.g., the starting flow rate, or a requested flow rate) satisfies the deviation threshold.

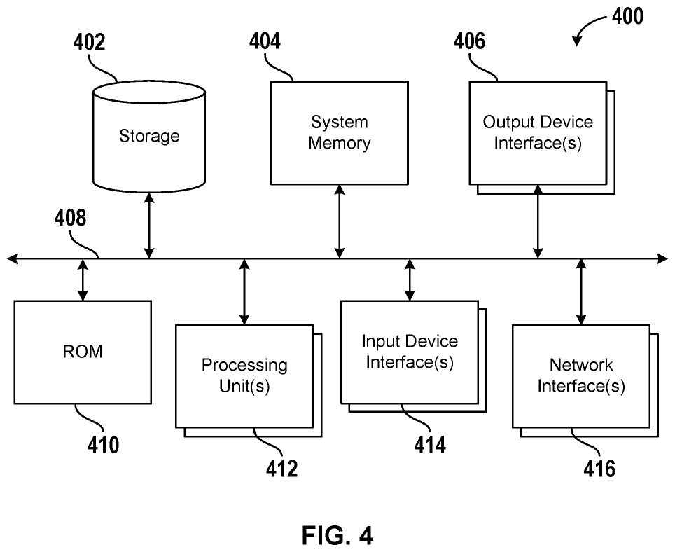

FIG. 4 is a conceptual diagram illustrating an example electronic system 400 for detecting a drop-factor identifier (e.g., the drop-factor identifier 214 of FIGS. 2A-2C) or a drip rate of an IV tubing set (e.g., the IV tubing set 200 of FIGS. 2A-2C), according to aspects of the subject technology. In some implementations, the electronic system 400 can also determine a drop factor and a flow rate of the IV tubing set based on the drop-factor identifier and the drip rate of the IV tubing set.

The electronic system 400 may be implemented by a computing device for execution of software associated with portions or steps of the processes discussed herein, or components and methods provided by FIGS. 1-3. In this regard, the electronic system 400 may include the device 100 or the display device 308. The electronic system 400 may also include a specifically-configured personal computer or a mobile device for infusion such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Additionally, the electronic system 400 may include various types of computer readable media and interfaces for various other types of computer readable media. In the depicted example, the electronic system 400 includes a bus 408, a processing unit(s) 412, a system memory 404, a read-only memory (ROM) 410, a permanent storage device 402, an input device interface(s) 414, an output device interface(s) 406, and a network interface(s) 416. In some implementations, the electronic system 400 may include or be integrated with other computing devices or circuitry for operation of the various components and methods previously described.

The bus 408 collectively represents all system, peripheral, and chipset buses that communicatively connect the numerous internal devices of the electronic system 400. For instance, the bus 408 communicatively connects the processing unit(s) 412 with the ROM 410, the system memory 404, and the permanent storage device 402.

From these various memory units, the processing unit(s) 412 retrieves instructions to execute and data to process in order to execute the processes of the subject disclosure. The processing unit(s) 412 can be a single processor or a multi-core processor in different implementations.

The ROM 410 stores static data and instructions that are needed by the processing unit(s) 412 and other modules of the electronic system. The permanent storage device 402, on the other hand, is a read-and-write memory device. This device is a non-volatile memory unit that stores instructions and data even when the electronic system 400 is powered off. Some implementations of the subject disclosure use a mass-storage device (such as a magnetic or optical disk and its corresponding disk drive) as the permanent storage device 402. Other implementations use a removable storage device (such as a floppy disk, or a flash drive and its corresponding disk drive) as the permanent storage device 402.

Like the permanent storage device 402, the system memory 404 is a read-and-write memory device. However, unlike the storage device 402, the system memory 404 is a volatile read-and-write memory, such as random-access memory (RAM). The system memory 404 stores some of the instructions and data that the processor needs at runtime. In some implementations, the processes of the subject disclosure are stored in the system memory 404, the permanent storage device 402, and/or the ROM 410. From these various memory units, the processing unit(s) 412 retrieves instructions to execute and data to process, in order to execute the processes of some implementations.

The bus 408 also connects to the input device interface(s) 414 and the output device interface(s) 406. The input device interface(s) 414 enables the user to communicate information and select commands to the electronic system. Input devices used with the input device interface(s) 414 include, for example, alphanumeric keyboards and pointing devices (also called “cursor control devices”). The output device interface(s) 406 enables, for example, the display of images generated by the electronic system 400. Output devices used with the output device interface(s) 406 include, for example, printers and display devices, such as cathode ray tubes (CRT) or liquid crystal displays (LCD). Some implementations include devices (e.g., touchscreens) that function as both input and output devices.

Furthermore, the bus 408 also couples the electronic system 400 to a network (not shown) through the network interface(s) 416. The network interface(s) 416 may include, for example, a wireless access point (e.g., Bluetooth or Wi-Fi) or radio circuitry for connecting to a wireless access point. The network interface(s) 416 may also include hardware (e.g., ethernet hardware) for connecting the computer to a part of a network of computers such as a local area network (LAN), a wide area network (WAN), wireless LAN, an intranet, or a network of networks, such as the Internet. Any or all components of the electronic system 400 can be used in conjunction with the subject disclosure when specifically configured with one of more of the features described.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field-programmable gate arrays (FPGAs). In some implementations, such integrated circuits execute instructions that are stored on the circuit itself.

As used in this specification and any claims of this application, the terms “computer,” “server,” “processor,” and “memory” all refer to electronic or other technological devices specifically configured with one or more of the features described above. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well. For example, feedback provided to the user can be any form of sensory feedback (e.g., visual feedback, auditory feedback, tactile feedback), and input from the user can be received in forms such as acoustic, speech, gesture, or tactile input. In addition, a computer can interact with a user by sending documents to and receiving documents from a device that is used by the user (e.g., by sending web pages to a web browser on a user's client device in response to requests received from the web browser).

Implementations of the subject matter described in this specification can be implemented in a specifically configured computing system that includes a back end component (e.g., a data server), or that includes a specifically configured middleware component (e.g., an application server), or that includes a specifically configured front end component (e.g., a client computer having a graphical user interface or a Web browser through which a user can interact with an implementation of the subject matter described in this specification), or any combination of one or more such back end, middleware, or front end components. The components of the system can be interconnected by one or more forms or mediums of digital data communication, such as a communication network. Examples of communication networks include a LAN and a WAN, an inter-network (e.g., the Internet), and peer-to-peer networks (e.g., ad hoc peer-to-peer networks).

The computing system can include specifically configured clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art will appreciate that the various illustrative blocks, modules, elements, components, methods, and algorithms described herein may be implemented as electronic hardware, computer software, or a combination thereof. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A device for detecting a drop-factor identifier of an intravenous (IV) tubing set, comprising: a housing having a recess configured to couple the device to a spike of an IV tubing set and receive a portion of a drip chamber connected to the spike while the device is coupled to the spike; a drop-factor sensor configured to detect a drop-factor identifier of the IV tubing set while the device is coupled to the spike via the recess, wherein the drop-factor identifier indicates a drop factor of the IV tubing set; and a drip-rate sensor configured to detect a drip rate of the IV tubing set while the device is coupled to the spike via the recess.

Clause 2. The device of clause 1, wherein: the drip chamber is cylindrical; and the recess is configured to circumscribe at least half of the portion of the drip chamber while receiving the portion of the drip chamber.

Clause 3. The device of any one of clauses 1 or 2, wherein: the recess comprises a first side and a second side opposite the first side; and the drip-rate sensor comprises a transmitter positioned in the first side of the recess and a receiver positioned in the second side of the recess such that a signal path between the transmitter and the receiver passes through the drip chamber.

Clause 4. The device of any one of clauses 1 through 3, wherein the recess comprises a shelf configured to sit on top of a base of the spike while the device is coupled to the spike.

Clause 5. The device of any one of clauses 1 through 4, wherein the device is configured to determine a flow rate of the IV tubing set based on dividing the drip rate by the drop factor.

Clause 6. The device of clause 5, wherein the device further comprises an onboard display, and the device is configured to display the flow rate via the onboard display.

Clause 7. The device of clause 6, wherein the device is configured to: display the drop factor via the onboard display; and receive a confirmation of the drop factor before displaying the flow rate via the onboard display.

Clause 8. The device of any one of clauses 5 through 7, further comprising a processor configured to determine a starting flow rate at a first time, determine a current flow rate at a second time subsequent to the first time, and trigger an alert in accordance with a determination that a difference between the current flow rate and the starting flow rate satisfies a deviation threshold.

Clause 9. The device of clause 8, wherein the processor is further configured to periodically determine, at a frequency based on the starting flow rate, the current flow rate and whether the difference between the current flow rate and the starting flow rate satisfies the deviation threshold.

Clause 10. The device of any one of clauses 8 or 9, wherein the device is configured to receive a confirmation of infusion prior to determining the starting flow rate at the first time.

Clause 11. The device of any one of clauses 1 through 10, wherein the drop-factor sensor comprises an optical sensor, the drop-factor identifier comprises a visual identifier, and detecting the drop-factor identifier comprises activating the optical sensor to detect a property of the visual identifier, wherein the visual identifier includes at least one of a color, a shape, a symbol, or an encoded glyph affixed to the portion of the drip chamber.

Clause 12. The device of any one of clauses 1 through 10, wherein the drop-factor sensor comprises a magnetic sensor, the drop-factor identifier comprises a magnetic identifier, and detecting the drop-factor identifier comprises activating the magnetic sensor to detect a property of a magnetic field proximate to the magnetic identifier, wherein the magnetic identifier includes shaped metal affixed to or embedded within the portion of the drip chamber.

Clause 13. A system for monitoring a flow rate of an IV tubing set, comprising: a device, comprising: a housing having a recess configured to couple the device to a spike of an IV tubing set and receive a portion of a drip chamber connected to the spike while the device is coupled to the spike; a drop-factor sensor configured to detect a drop-factor identifier of the IV tubing set, wherein the drop-factor identifier indicates a drop factor of the IV tubing set; a drip-rate sensor configured to detect a drip rate of the IV tubing set; and a processor communicatively coupled with the device and configured to determine a flow rate of the IV tubing set based at least in part on the drop-factor identifier and the drip rate; and a display device communicatively coupled with the processor and configured to receive the flow rate from the processor and display the flow rate.

Clause 14. The system of clause 13, wherein the processor is configured to determine the drop factor based on the drop-factor identifier, determine the flow rate based on dividing the drip rate by the drop factor, and provide the flow rate to the display device.

Clause 15. The system of any one of clauses 13 or 14, wherein the device is a first device and the system further comprises a second device comprising: a second recess configured to couple the second device to another spike of another IV tubing set and receive a portion of another drip chamber connected to the other spike while the second device is coupled to the other spike; a second drop-factor sensor configured to detect another drop-factor identifier of the other IV tubing set while the second device is coupled to the other spike via the second recess, wherein the other drop-factor identifier indicates another drop factor of the other IV tubing set; and a second drip-rate sensor configured to detect another drip rate of the other IV tubing set.

Clause 16. The system of clause 15, wherein the second device is configured to: determine another flow rate of the other IV tubing set based on dividing the other drip rate by the other drop-factor; and provide, for display on the display device, the other flow rate.

Clause 17. The system of clause 13, wherein the processor is configured to: display the drop factor via the display device or an onboard display of the device; and receive a confirmation of the drop factor before displaying the flow rate via the display device or the onboard display.

Clause 18. The system of clause 17, wherein the processor is configured to determine a starting flow rate at a first time, determine a current flow rate at a second time subsequent to the first time, and trigger an alert in accordance with a determination that a difference between the current flow rate and the starting flow rate satisfies a deviation threshold.

Clause 19. The system of clause 18, wherein the processor is configured to periodically determine, at a frequency based on the starting flow rate, the current flow rate and whether the difference between the current flow rate and the starting flow rate satisfies the deviation threshold.

Clause 20. The system of any one of clauses 18 or 19, wherein the processor is configured to receive a confirmation of infusion prior to determining the starting flow rate at the first time.

### Further Consideration:

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects.

Thus, the claims are not intended to be limited to the aspects shown herein but are to be accorded the full scope consistent with the language of the claims. For example, reference to an element in the singular is not intended to mean “one and only one” unless specifically so stated, but rather “one or more.” Moreover, unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to,” “operable to,” and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term “automatic,” as used herein, may include performance by a computer or machine without user intervention, for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface, or a UI) may refer to a network-based interface including data fields or other control elements for receiving input signals or providing electronic information or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database, or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine-readable aggregation of information such as an XML document, fixed field message, comma separated message, JSON, a custom mode, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fu5y logic, pattern matching, a machine learning assessment model, or combinations thereof.

In some implementations, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

1. A device for detecting a drop-factor identifier of an intravenous (IV) tubing set, comprising:
a housing having a recess configured to couple the device to a spike of an IV tubing set and receive a portion of a drip chamber connected to the spike while the device is coupled to the spike;
a drop-factor sensor configured to detect a drop-factor identifier of the IV tubing set while the device is coupled to the spike via the recess, wherein the drop-factor identifier indicates a drop factor of the IV tubing set; and
a drip-rate sensor configured to detect a drip rate of the IV tubing set while the device is coupled to the spike via the recess.

2. The device of claim 1, wherein:
the drip chamber is cylindrical; and
the recess is configured to circumscribe at least half of the portion of the drip chamber while receiving the portion of the drip chamber.

3. The device of claim 1, wherein:
the recess comprises a first side and a second side opposite the first side; and
the drip-rate sensor comprises a transmitter positioned in the first side of the recess and a receiver positioned in the second side of the recess such that a signal path between the transmitter and the receiver passes through the drip chamber.

4. The device of claim 1, wherein the recess comprises a shelf configured to sit on top of a base of the spike while the device is coupled to the spike.

5. The device of claim 1, wherein the device is configured to determine a flow rate of the IV tubing set based on dividing the drip rate of the IV tubing set by the drop factor of the IV tubing set.

6. The device of claim 5, wherein the device further comprises an onboard display, and the device is configured to display the flow rate via the onboard display.

7. The device of claim 6, wherein the device is configured to:
display the drop factor via the onboard display; and
receive a confirmation of the drop factor before displaying the flow rate via the onboard display.

8. The device of claim 5, further comprising a processor configured to determine a starting flow rate at a first time, determine a current flow rate at a second time subsequent to the first time, and trigger an alert in accordance with a determination that a difference between the current flow rate and the starting flow rate satisfies a deviation threshold.

9. The device of claim 8, wherein the processor is further configured to periodically determine, at a frequency based on the starting flow rate, the current flow rate and whether the difference between the current flow rate and the starting flow rate satisfies the deviation threshold.

10. The device of claim 8, wherein the device is configured to receive a confirmation of infusion prior to determining the starting flow rate at the first time.

11. The device of claim 1, wherein the drop-factor sensor comprises an optical sensor, the drop-factor identifier comprises a visual identifier, and detecting the drop-factor identifier comprises activating the optical sensor to detect a property of the visual identifier, wherein the visual identifier includes at least one of a color, a shape, a symbol, or an encoded glyph affixed to the portion of the drip chamber.

12. The device of claim 1, wherein the drop-factor sensor comprises a magnetic sensor, the drop-factor identifier comprises a magnetic identifier, and detecting the drop-factor identifier comprises activating the magnetic sensor to detect a property of a magnetic field proximate to the magnetic identifier, wherein the magnetic identifier includes shaped metal affixed to or embedded within the portion of the drip chamber.

13. A system for monitoring a flow rate of an IV tubing set, comprising:
a device, comprising:
a housing having a recess configured to couple the device to a spike of an IV tubing set and receive a portion of a drip chamber connected to the spike while the device is coupled to the spike;
a drop-factor sensor configured to detect a drop-factor identifier of the IV tubing set, wherein the drop-factor identifier indicates a drop factor of the IV tubing set;
a drip-rate sensor configured to detect a drip rate of the IV tubing set; and
a processor communicatively coupled with the device and configured to determine a flow rate of the IV tubing set based at least in part on the drop-factor identifier and the drip rate; and
a display device communicatively coupled with the processor and configured to receive the flow rate from the processor and display the flow rate.

14. The system of claim 13, wherein the processor is configured to determine the drop factor based on the drop-factor identifier, determine the flow rate based on dividing the drip rate by the drop factor, and provide the flow rate to the display device.

15. The system of claim 13, wherein the device is a first device and the system further comprises a second device comprising:
a second recess configured to couple the second device to another spike of another IV tubing set and receive a portion of another drip chamber connected to the other spike while the second device is coupled to the other spike;
a second drop-factor sensor configured to detect another drop-factor identifier of the other IV tubing set while the second device is coupled to the other spike via the second recess, wherein the other drop-factor identifier indicates another drop factor of the other IV tubing set; and
a second drip-rate sensor configured to detect another drip rate of the other IV tubing set.

16. The system of claim 15, wherein the second device is configured to:
determine another flow rate of the other IV tubing set based on dividing the other drip rate by the other drop-factor; and
provide, for display on the display device, the other flow rate.

17. The system of claim 13, wherein the processor is configured to:
display the drop factor via the display device or an onboard display of the device; and
receive a confirmation of the drop factor before displaying the flow rate via the display device or the onboard display.

18. The system of claim 17, wherein the processor is configured to determine a starting flow rate at a first time, determine a current flow rate at a second time subsequent to the first time, and trigger an alert in accordance with a determination that a difference between the current flow rate and the starting flow rate satisfies a deviation threshold.

19. The system of claim 18, wherein the processor is configured to periodically determine, at a frequency based on the starting flow rate, the current flow rate and whether the difference between the current flow rate and the starting flow rate satisfies the deviation threshold.

20. The system of claim 18, wherein the processor is configured to receive a confirmation of infusion prior to determining the starting flow rate at the first time.
